(19) United States
(12) Patent Application Publication
(10) Pub. No.: US 2008/0051630 A1
(43) Pub. Date: Feb. 28, 2008

(54) Endoscopic Lubricating And Gripping Device

(76) Inventors: John M. Levey, Westborough, MA (US); Jennifer Dziczek, Boylston, MA (US)

Correspondence Address: Cahill, von Hellens & Glazer P.L.C., 155 Park One, 2141 E. Highland Avenue, Phoenix, AZ, 85016, US

(21) Appl. No.: 10/583,292

(22) PCT Filed: Dec. 14, 2004
(86) PCT No.: PCT/US04/41830

§ 371 Date: Mar. 27, 2007

Related U.S. Application Data
(60) Provisional application No. 60/529,558, filed on Dec. 16, 2003.
(60) Provisional application No. 60/599,085, filed on Aug. 6, 2004.

(51) Int. Cl. A61B 1/04

(52) U.S. Cl. 600/114

(57) ABSTRACT

A non-invasive device for lubricating and facilitating the insertion and removal of an endoscope into a body cavity. In one embodiment, the device consists of an annular outer tube having an inner diameter sized to permit sliding of an endoscope therethrough. The outer surface of the outer tube may be suitably textured for improved gripping. A foam annular tube is positioned within the outer tube, with an inner diameter slightly less than the normal outer diameter of the endoscope so that passage of the endoscope compresses the foam as it is passed through. The foam tube may be saturated with a lubricant which is forced from the foam to coat the endoscope as it moves through. Flanges positioned respectively at the distal and proximal ends of the tube provide a barrier to movement of excess lubricant onto the central portion of the tube. The invention also comprises the use of a reservoir for lubricants to be applied to the endoscope as it passes through the device.

### RELATED APPLICATIONS

This application claims priority under 35 U.S.C. § 119(e) to U.S. Provisional Application Ser. No. 60/599,085, entitled “ENDOSCOPIC LUBRICATING AND GRIPPING DEVICE,” filed on Aug. 4, 2004, and to U.S. Provisional Application Ser. No. 60/529,558, entitled “ENDOSCOPIC LUBRICATING DEVICE,” filed on Dec. 16, 2003, which are herein incorporated by reference in their entirety.

### FIELD OF INVENTION

The present invention relates generally to a non-invasive device for lubricating and facilitating the insertion and removal of an endoscope into a body cavity.

### BACKGROUND OF INVENTION

Endoscopes used for examining body cavities are inserted through a body orifice. Such insertions generally involve coating the endoscope surface with a suitable lubricant, such as a petroleum-based lubricant. For a long time, lubricants have been applied manually to the endoscope before or during insertion. This manual method has drawbacks since lubricating the endoscope by hand while simultaneously controlling and moving it through the orifice is time consuming, messy and not always effectively performed. Additionally, lubricating by hand frequently results in an unsatisfactory coating since much more lubricant than may be required is deposited on the endoscope.

Heretofore, attempts have been made to apply a lubricant to the endoscope tube using appropriate attachments. Typical of these are attachments shown in the Fukuda et. al U.S. Pat. No. 3,871,358 and the Okada U.S. Pat. No. 3,805,770. Both of these devices are tubular shaped guides which are inserted at least partially into the large intestines through the sphincter. Since these devices must have larger diameters than the endoscope, they apply more pressure and are likely to cause more distress to the patient than would be the case in which only the endoscope is inserted.

### SUMMARY OF INVENTION

The present invention provides a novel device that has the dual function of both lubricating and facilitating the controlled insertion and removal of the endoscope tube through the body cavity of any patient. This invention provides an improved means for coating the endoscope as it is being inserted while contemporaneously avoiding increased distress on a patient by avoiding the insertion of the control itself into the body cavity.

In the present invention, there is provided a non-invasive external control or device for both lubricating and facilitating the insertion and removal of the endoscope into the body cavity. In this arrangement, an annular compressible tube having an outer and inner surface with the inner surface of the tube sized to permit sliding passage there through of an endoscope. The annular tube is provided with means for coating the endoscope in the form of a compressible reservoir or foam tube. The tube is coaxial within and at least partially co-extensive with the outer tube. The foam tube may be appropriately impregnated with a lubricant from the reservoir or suitably pre-impregnated for the procedure. The foam tube has an inner diameter in a sliding fit with the outer diameter of the endoscope which is inserted so that the endoscope, as moved through, has lubricant impregnated in the foam tube deposited onto the endoscope surface when the annular tube is squeezed or compressed. Additionally, an outwardly extending flange adjacent to the proximal end of the control may be provided with a plurality of radially extended passages for insertion of additional lubricant, with these passages communicating directly with the foam within the outer tube.

The present invention also contemplates the use of a device for facilitating insertion of an endoscope tube into a body cavity. In this method, the endoscope tube is passed through an externally positioned control containing a lubricant which is deposited on the endoscope surface as it passes through the control.

### BRIEF DESCRIPTION OF DRAWINGS

These and other objects and advantages of the present invention may be more fully considered in connection with the accompanying drawings in which:

FIG. 1 is a perspective view of a control embodying a preferred embodiment of the present invention;

FIG. 2 is an end view thereof along the line 2-2 of FIG. 1;

FIG. 3 is an end view thereof along the line 3-3 of FIG. 1,

FIG. 4 is a cross-sectional view taken along the line 4-4 of FIG. 2;

FIG. 5 is a perspective view of another embodiment of the invention;

FIG. 6 is an end view along the line 6-6 of FIG. 5;

FIG. 7 is an end view along the line 7-7 of FIG. 5;

FIG. 8 is a cross-sectional view taken along the line 8-8 of FIG. 6; and

FIG. 9 is a cross-sectional view of a further embodiment of the present invention.

### DETAILED DESCRIPTION

The foregoing advantages of the present invention will be more fully understood when considered in conjunction with detailed description of the present invention.

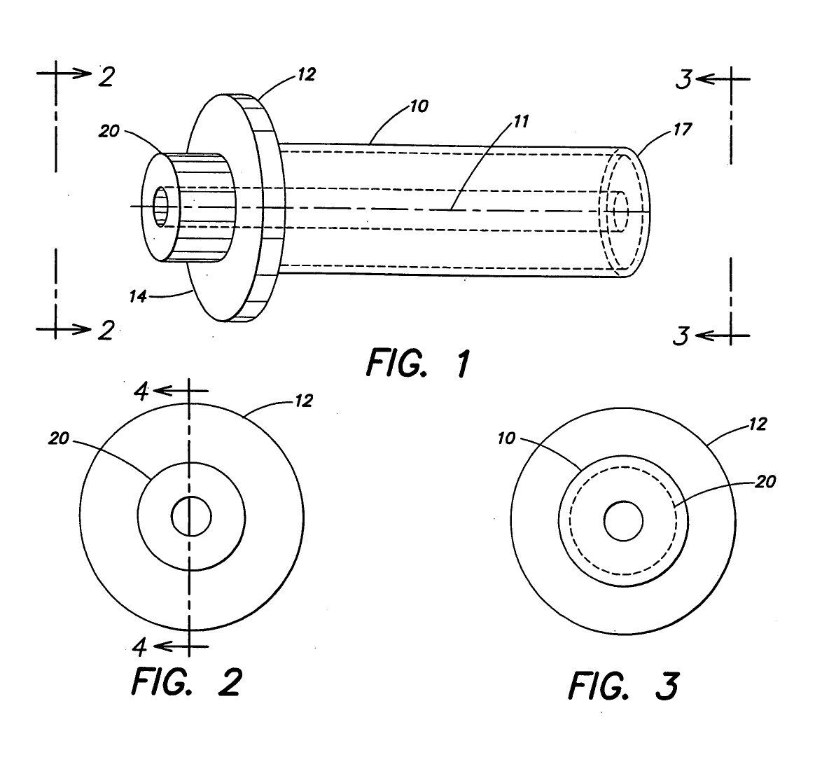

Referring first to FIG. 1, there is illustrated a squeezable outer tube 10 having a length in the order of 4″ to 8″ or sufficiently long to permit an operator to grip the tube 10 in one hand. The tube should be made of a suitable, plastic that has sufficient flexibility to permit the operator to grasp the tube and apply pressure therethrough onto the endoscope to control its movement and cause lubricant to be deposited on the endoscope. In a preferred embodiment, a reservoir 12 extends radially from the tube 10 at the exit (or proximal) end 14. The reservoir 12 is positioned close to or at the proximal end 14 of the tube 10. The reservoir may be integrally molded with the tube 10. The reservoir has an outer diameter sized so that it will not ordinarily permit movement of the device into the body cavity.

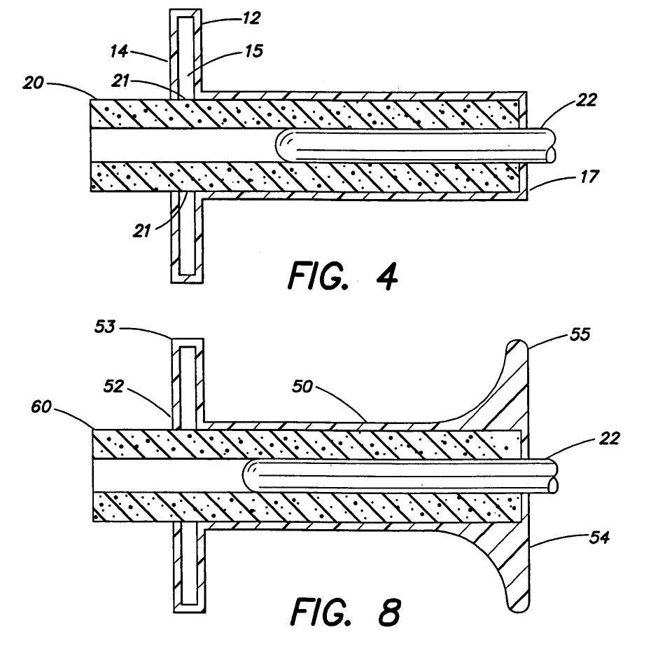

Positioned within the tube 10 is a flexible, resilient foam annular tube 20 having an outer surface engaging the inner surface of the tube 10 (FIG. 4). The foam tube 20 has an inner diameter that is in sliding contact with the outer diameter of the endoscope tube 22 to be used with it so that the flexible tube 20 will normally facilitate deposition of lubricant onto the endoscope tube 22 as it slides through the interior of the tube 20 when tube 10 is squeezed.

The reservoir 12 is loaded with a suitable lubricant such as a petroleum-based jelly or any other lubricant commonly used for insertion of endoscopes tube through the body cavity.

The amount of lubricant pre-loaded into the reservoir 12 may vary depending upon the particular uses to which the control is placed. Additional lubricants may be suitably loaded into foam tube 20 with the lubricant distributed through the foam tube 20. The inner wall of the reservoir 12 is preferable coincident with the inner surface of tube 10 and is provided with a series of openings 21 to permit passage of lubricant onto the tube 20. If desired, radially extending passages may be formed in the foam tube 20 in alignment with cavity 15 so that the lubricant may pass through the foam tube 20 directly onto an endoscope tube or into the interior of the flexible tube 10.

The flange 12 which defines the cavity 15 may be formed of a flexible thin plastic walls capable of being physically deflected by an operator to force any lubricant within the cavity 15 into and through the tube 20. The tube 20 as noted is preferably formed of a foam plastic which may be either open or closed cell provided it will permit absorption of lubricant and subsequent movement of the lubricant from the foam material when compressed onto the endoscope tube 22. As illustrated, the foam tube 20 preferably projects a short distance beyond the proximal end 14 of the tube so that lubricant contained within reservoir 12 will be deposited on the inner surface of this projecting end of the tube 20 as the endoscope tube 22 moves through. The distal end of the control is formed with an inwardly extending flange defining the distal end 17. This flange provides a limiting support enclosure for the distal end of the flexible tube 20. The flange at the distal end 17 is formed with an opening just sufficient to permit passage through it of the endoscope tube 22.

In using the control shown in FIGS. 1-4 to insert an endoscope into a body cavity the operator first threads the endoscope tube 22 through the opening in the proximal end 17 into the tube 20. The tube 20 is slightly compressed as the endoscope tube 22 moves through it thus forcing petroleum jelly that may have been loaded into the tube 20 into intimate contact with the outer surface of the endoscope tube 22 immediately prior to insertion through the body cavity. The operator may also flex or thereby squeeze or otherwise force lubricant contained in the reservoir 12 through the walls of the tube 20 onto the outer surface of the endoscope tube 22 prior to its insertion. The reservoir 12 serves a dual purpose of preventing insertion of the control into the body cavity. It also further provides a barrier for preventing excess lubricant from moving backward onto the outer surface of the control 10 in contact with the operator's hand.

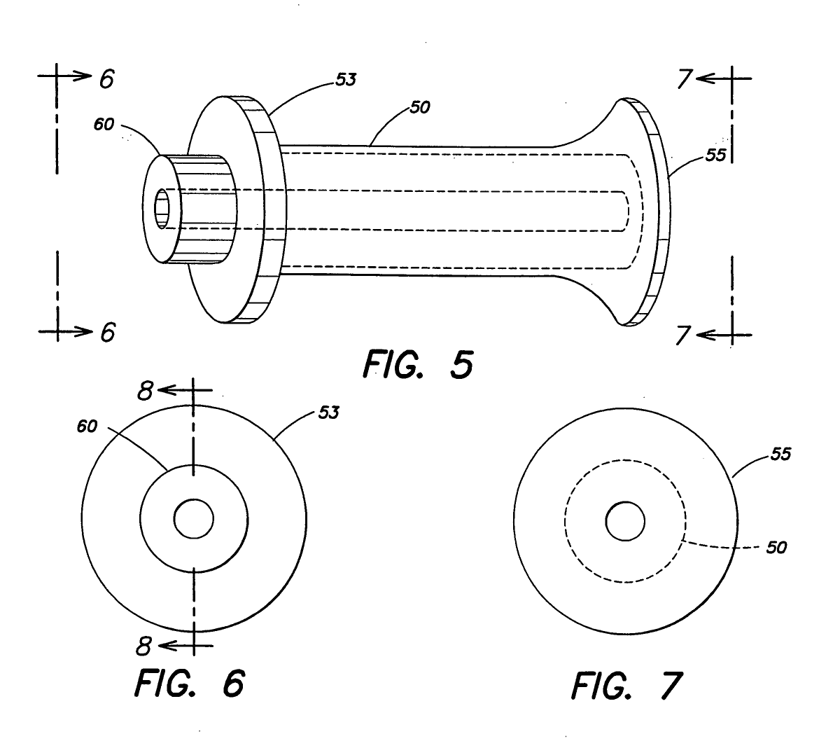

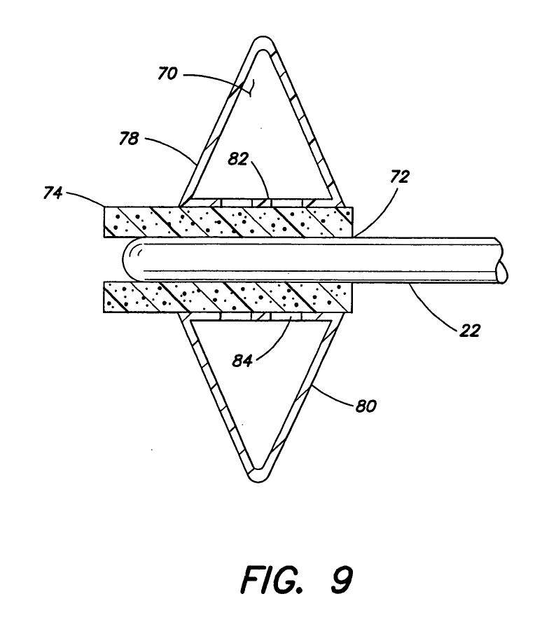

The embodiment shown in FIGS. 5-8 is generally similar in some respects to the previously described embodiment with the principal exception that a flange is positioned at the distal end of the control to limit the amount of excess lubricant that will contact the operator's hand while it is on the control. In this embodiment, tube 50 is similar in material and construction to tube 10 of the preferred embodiment. Tube 50 is preferably long enough to comfortably accommodate the hand of an operator. Presumably this will involve a tube on the order of four to eight inches long. The tube 50 terminates at its proximal end 52 in reservoir 53 and at its distal end 54 at flange 55. This reservoir 53 and flange 55 may be integrally formed with the tube 50 and extend radially outwardly from either end to define a barrier to movement of lubricants onto the central portion of the tube 50. Flange 55 may have a variety of forms. As illustrated, the flange has essentially a frusto conic shape for ease in gripping. Other shapes such as a planer flange are also contemplated.

Positioned within the tube 50 is a foam tube 60 similar in construction to the foam tube shown in FIGS. 1-4. Foam tube 60 is coaxial with and positioned within tube 50 with the outer surface of tube 60 in intimate contact with the inner surface of tube 50. The tube 60 may be of varied length. In the embodiment shown in FIGS. 5-8 the tube 60 extends from the distal end 54 beyond the proximal end 52 for a short distance. This distance may vary depending upon the particular needs and interests for which the unit is used. The invention also contemplates the use of a foam tube 60 which is positioned within tube 50 but not for its full length. The flange 55 at the distal end of the tube 50 may extend radially inwardly of the inner wall of tube 50 to provide an annular support for the distal end of the tube 60 as illustrated in FIG. 8. The reservoir 53 functions in a manner similar to the function of reservoir 12 in the embodiment of FIG. 1.

In an alternative embodiment to FIG. 1, the outer tube 10 and the foam tube 20 may be longitudinally cut along a line 11. The cut may be continuous or a line of perforations such that the control may be removed from an endoscope while the endoscope is inserted into the body cavity. In such a procedure the control is split apart along line 11 and removed from the endoscope. Since both the outer tube 10 and foam tube 20 are flexible, it may be done with ease. Conversely, a fresh control may be applied to the endoscope by spreading the unit along line 11 and snapping it over the endoscope. Such unit should be made of a resilient plastic to permit the unit to be flexed open to fit around the endoscope tube to retain its shape when so positioned.

The embodiment illustrated in FIGS. 5-8 is used in a fashion similar to the embodiment in FIGS. 1-4. The embodiment of FIGS. 5-8 may be further modified to eliminate the reservoir 53 and substitute a simple flange. In this embodiment the lubricant must be fully loaded into the foam tube 60 and must have sufficient concentration to adequately coat the endoscope tube 22 as it passes through the control. In addition to providing an adequate volume of lubricant within the foam tube 60 the inner diameter of tube 60, the density of the foam and other parameters should be selected so as to provide an adequate coating of lubricant onto the tube 22 as it moves through the control.

Referring now to the embodiment of FIG. 9, there is illustrated a device in which lubricant is contained in a chamber 70. Chamber 70 may have various shapes. In the particular embodiment shown, it compresses an annular container defining an opening 72 therethrough sized to permit the passage of an endoscope tube 22. The opening is lined with a foam tube 74 similar in composition and diameter to tube 20. The tube 74, however, extends only a short distance through the opening 72. The shape of the chamber 70 illustrated is polyhedral with inner circular wall 78 and outer wall 80. The circular wall 82 is provided with a plurality of perforations 84 for passage of the lubricant in the chamber into the tube 74. The chamber walls are compressible to force lubricant from the chamber through openings 84. In use, the operator threads the endoscope tube through opening 72 and forces lubricant in the chamber 70 into the tube 74 from which it coats the tube 22 and it is moved through the device.

Having thus described at least one illustrative embodiment of the invention, various alterations, modifications, and improvements will readily occur to those skilled in the art. Such alterations, modifications, and improvements are intended to be within and scope of the invention. Accordingly, the foregoing description is by way of example only and is not intended as limiting. The invention is limited only as defined in the following claims and the equivalents thereto.

1. A non-invasive external control for facilitating the insertion and removal of an endoscope into a body cavity comprising:
an annular tube having an outer and inner surface with the inner diameter of the tube sized to permit sliding passage therethrough of the insertion end of an endoscope tube,
a first flange extending outwardly from the annular tube closer to the proximal end thereof, wherein the diameter of the first flange is sized to preclude insertion of the control into the body cavity of a patient,
a second flange extending outwardly from the annular tube closer to the distal end thereof,
said annular tube having means for coating the endoscope tube as it passes through the annular tube with a lubricants
wherein the first and second flanges act as barriers to prevent lubricant from migrating onto said endoscope control.

2. (canceled)

3. A control as set forth in claim 1, wherein said coating means comprises a compressible foam member impregnated with a lubricant positioned to engage an endoscope tube as it moves through the control.

4. A control as set forth in claim 3, wherein the foam member comprises an annular tube at least partially positioned at one end within the annular tube and having an inner uncompressed diameter substantially equal to the outer diameter of an endoscope tube intended to be controlled.

5-7. (canceled)

8. A control as set forth in claim 1, wherein the distance between the first and second flanges provide a sufficient length along the annular tube for hand-gripping.

9. A control as set forth in claim 1, wherein said first flange has a passage extending radially from the inner surface of said annular tube outwardly through the annular flange, wherein said passage feeds a lubricant into the interior of the annular tube.

10. A control as set forth in claim 9, wherein said passage extending radially to the outer periphery of said first flange.

11. (canceled)

12. A control as set forth in claim 3, wherein the compressible foam member comprises an annular tube having a major portion of its length coaxial and coextensive with at least a portion of said annular tube.

13. A method of inserting, removing, and controlling an endoscope into a body cavity comprising:
providing an endoscope for insertion into a body cavity;
providing an endoscope control which includes a tubular member having a first flange at the proximal end of the tubular member, and a second flange at the distal end of the tubular member, wherein said first flange prevents any portion of the endoscope control from entering the body cavity:
threading the leading end of the endoscope through the endoscope control;
providing a quantity of lubricant positioned within the endoscope control to coat the leading end of the endoscope with the lubricant immediately before insertion of the endoscope into the body, wherein said first and second flanges act as barriers to prevent lubricant from migrating onto said endoscope control; and
inserting the endoscope into a body cavity and thereafter at least partially directing the endoscope as it moves into the body cavity by manual control of the endoscope control with the endoscope control entirely external of the body cavity.

14. A control as set forth in claim 12, wherein the foam annular tube is positioned interior the annular tube.

15. A control as set forth in claim 3 wherein the control has means for opening the control along its length.

16. A control as set forth in claim 1, wherein said second flange is shaped for gripping to assist in controlling the insertion and removal of the endoscope tube.

17. A control as set forth in claim 1, wherein said annular tube is compressible to control said means for coating t he endoscope tube with a lubricant.

18. A control as set forth in claim 1, further comprising a reservoir for holding the lubricant, wherein said reservoir is located within said first flange.

19. The method as recited in claim 13, wherein said second flange is shaped for gripping to assist in the manual control of said endoscope device.

20. The method as recited in claim 13, further comprising:
providing a reservoir for holding the lubricant, wherein said reservoir is located within said first flange.

21. The method as recited in claim 13, further comprising:
squeezing said tubular member to control the quantity of lubricant coated on said endoscope.
